SEQUENCE LISTING (4 sequences)

SEQ ID NO: 1 - DNA, 1113 bp, synthetic construct
  source 1..1113: mol_type = other DNA
atggccgccgagcatgccaccgccgcggtgggtgagccgcctcccgccacgtcgcagccggcggagggcgtgacggcggcggcgggccagaggtccgtgcccacgccgttcctcagcaagacgtaccagctggtggacgacccggcggtggacgacatcatctcgtggaacgacgacgggtctgccttcatcgtgtggcgcccggcggagttcgcgcgcgatctgttgcccaagtacttcaagcacaacaacttctccagcttcgtgcgccagctcaacacctacgggttcaggaagatcgtgccggacaggtgggagttcgcgaacgactgcttccgccgaggggagaagcggctgctctgcgacatacaccgccggaaggtgtcgccggccacgggggcggtcacggtggccgccgccgctgcggccatcccgatggcgctgcccgtgggctctccggtgtactccggcgaggagcaggtgctgtcaagctcgtccccggagcccccgtcgctgcagcagcacccgccggcgccgtccgggtcgggctccggcggggtggtctcgggcgacgtgggcgaggagaacgagcggttgcggcgggagaacgcgcggctggcgcgcgagctcggacagatgaagaagctctgcaacaacatcctcctcctcatgtccaagtacgccgccactcagcagccggacgccgccaaggaggcggccgccgggaactgcaccggcgagtcgtcggatgccgccgcggcgcctccgctcccttcgatcctggagctcctaccctcgtgccgcgccgaccccgcgcccgctgcagccggaacggatcacgaggatgacgaaaaggcgggcgctaggctgttcggcgtctccatcgggcggaaacgaatgcgggacgagagcgaccaccacgccggcgtctgcgcagcggaggtgaaagccgagccggtggacgcgcgtcccgaccagcagcggcagcgcaacgccacggagccgcagtcgtggcccatttaccggcccaggcctgtgtaccacccgctccgggcctgcaacggctcgggcagcgctgggagcgaccacgacgggtccaacgactcgaggtga

SEQ ID NO: 2 - protein, 370 aa, synthetic construct
MAAEHATAAVGEPPPATSQPAEGVTAAAGQRSVPTPFLSKTYQLVDDPAVDDIISWNDDGSAFIVWRPAEFARDLLPKYFKHNNFSSFVRQLNTYGFRKIVPDRWEFANDCFRRGEKRLLCDIHRRKVSPATGAVTVAAAAAAIPMALPVGSPVYSGEEQVLSSSSPEPPSLQQHPPAPSGSGSGGVVSGDVGEENERLRRENARLARELGQMKKLCNNILLLMSKYAATQQPDAAKEAAAGNCTGESSDAAAAPPLPSILELLPSCRADPAPAAAGTDHEDDEKAGARLFGVSIGRKRMRDESDHHAGVCAAEVKAEPVDARPDQQRQRNATEPQSWPIYRPRPVYHPLRACNGSGSAGSDHDGSNDSR

SEQ ID NO: 3 - DNA, 19 bp, synthetic construct
  source 1..19: mol_type = other DNA
atggccgccgagcatgcca

SEQ ID NO: 4 - DNA, 21 bp, synthetic construct
  source 1..21: mol_type = other DNA
tcacctcgagtcgttggaccc

(19) United States
(12) Patent Application Publication
(10) Pub. No.: US 2023/0272411 A1
(43) Pub. Date: Aug. 31, 2023

(54) HEAT-SHOCK RELATED GENE ZMHSF11 AND APPLICATION OF ZMHSF11 IN REGULATING HEAT-RESISTENCE OF PLANT

(71) Applicant: ANHUI AGRICULTURAL UNIVERSITY, HEFEI (CN)

(72) Inventors: HAIYANG JIANG, Hefei (CN); QIANQIAN QIN, Hefei (CN); WEINA SI, Hefei (CN); YUJUN ZHAO, Hefei (CN); XIAOJIAN PENG, Hefei (CN)

(21) Appl. No.: 18/181,561

(22) Filed: Mar. 10, 2023

Related U.S. Application Data
(63) Continuation of application No. PCT/CN2022/090924, filed on May 5, 2022.

(30) Foreign Application Priority Data
May 27, 2021 (CN) 202110582029.5

(51) Int. Cl. C12N 15/82; A01H 6/46; C07K 14/415

(52) U.S. Cl. CPC C12N 15/8271; C12N 15/8218; A01H 6/4684; A01H 6/4636; C07K 14/415

(57) ABSTRACT

The present disclosure provides a heat-stress related gene ZmHsf11 and an application of the gene in regulating heat resistance of plants. The present disclosure belongs to the field of biotechnology. The present disclosure reports for the first time a heat resistance negative regulation related gene ZmHsf11 and its protein. In the present invention, it is found that the expression of ZmHsf11 gene can be induced by adverse stresses such as high temperatures. By overexpressing the gene in rice and identifying functions of the gene, it is found that a survival rate of the overexpressed rice plant under the heat treatment is significantly reduced after overexpression. By identifying functions of the ZmHsf11 mutant plant obtained in the present invention, the heat resistance of the mutant plant is significantly increased after the heat treatment.

### CROSS REFERENCE TO RELATED APPLICATIONS

The present application is a continuation-application of the International Patent Application No. PCT/CN2022/090924, filed on May 5, 2022, which claims the priority of the Chinese Patent Application No. 202110582029.5, filed on May 27, 2021, in the China National Intellectual Property Administration, and the entire contents of which are hereby incorporated by reference in their entireties.

### SEQUENCE LISTING

The instant application contains a Sequence Listing which has been submitted electronically in ST.26 format and is hereby incorporated by reference in its entirety. The ST.26 copy, created on Feb. 2, 2023, is named Sequence Listing.xml and is 6 kilobytes in size.

### TECHNICAL FIELD

The present disclosure relates to the field of molecular biology, and in particular relates to a heat-shock related gene ZmHsf11, isolation and identification of a protein encoded by the heat-shock related gene ZmHsf11, a method for obtaining a genomic sequence of the heat-shock related gene ZmHsf11, and an application of the heat-shock related gene ZmHsf11 in regulating heat-resistance of plants.

### BACKGROUND

Maizes serves as one of the important food-feed crops in China. A slight change in an environment can reduce yield and quality of the maizes. Currently, environmental factors, mainly including biotic and abiotic stresses, can influence growth and development of the maizes. Problems caused by the biotic stresses can be solved by applying herbicides and pesticides, and so on. The abiotic stresses include high temperatures, droughts, frost damages, and so on, and cannot be solved effectively. Especially for crops, each of these stresses can cause low or extinction yields of the crop (Liu et al., 2016).

A responsive molecular mechanism of the crops under the abiotic stresses is explored to find an advantageous trait gene to improve the yield and the quality of the crops. In addition, as the modern molecular technology and molecular genetic breeding develop rapidly, the advantageous gene may be transfected into and stably expressed in an advantageous strain. In this way, tolerance of the crops under adverse stresses may be improved. Therefore, the study of resistance genes and breeding of new species having advantageous traits have become one of hot topics in plant resistance genetic breeding research.

Plants are immobilized organisms, and the high-temperature stress has a great impact on their growth, development and reproduction. For no beneficial tropism, plants can only achieve homeostasis by regulating an internal environment to resist an external environment. When the plant is subjected to a high temperature transiently, expression of some heat-shock genes is activated in vivo so that the plant may generate acquired heat-resistance once growth is resumed. For basal heat-resistance, when the plant is subjected to the high temperature stress, some in vivo genes may regulate the homeostasis of the internal environment of the plant to be resistant to the external environment. The above process involves expression of certain genes, such as genes of heat shock proteins. The genes that have been reported to affect the heat-resistance of plants include genes related to the phytohormone or signaling pathways, genes related to Ca2+ signaling pathways, genes related to reactive oxygens, and genes of heat-shock transcription factors and heat-shock protein genes that play a major role (Mittler et al., 2012).

The heat shock transcription factor (HSF) is a major transduction element in a heat stress signaling pathway. The HSF activates expression of related genes under the heat stress and plays a critical role in regulation of heat stress response processes in plants (Kotak et al., 2007). The HSF family has three major sub-families: A, B, and C. Proteins expressed by the three major sub-families have similar structures, but these structures may differ from each other across the three major sub-families. For example, some members of the B sub-family may further have a repressor region (RD) (Nover et al., 2001). Therefore, functions of different HSF members may be involved in a wide variety of adverse stresses. Current studies have shown that the functional studies of the HSF are mainly focused on stresses such as droughts, salts, and high temperatures.

### SUMMARY OF THE DISCLOSURE

The present disclosure aims to screen and identify ZmHsf11, a heat-shock related gene capable of responding to high temperatures and an application of the ZmHsf11 in regulating heat resistance of plants.

In order to achieve the above aim, the present disclosure provides the following technical solutions.

The present disclosure provides a heat-stress related gene ZmHsf11. A nucleotide sequence of the heat-stress related gene ZmHsf11 is shown as SEQ ID No: 1, and the heat-stress related gene ZmHsf11 has function of reducing heat resistance in plants.

The present disclosure provides a protein encoded by the heat-stress related gene ZmHsf11. The protein is the following (1) and (2) proteins.

(1) A protein consists of an amino acid sequence as shown in SEQ ID No: 2 in the sequence listing.

(2) A protein is derived from the protein (1) by substituting and/or adding one to ten amino acid residues to the amino acid sequence of SEQ ID No: 2 in the sequence listing, and has the function of the heat-shock related gene ZmHsf11.

The present disclosure provides a plant expression vector for the heat-shock related gene ZmHsf11.

Further, a nucleic acid molecule of the heat-shock related gene ZmHsf11 is inserted into an expression vector p1301a, and a vector for overexpressing the ZmHsf11, p1301a-ZmHsf11, is obtained.

The present disclosure provides a host bacterium of the plant expression vector.

The present disclosure provides a plant cell including the heat-shock related gene ZmHsf11, a plant including the plant cell, and a seed of the plant, and the plant is preferably rice or maize.

The present disclosure provides a primer pair for cloning the heat-shock related gene ZmHsf11. The primer pair includes an upstream primer and a downstream primer. A nucleotide sequence of the upstream primer is shown in SEQ ID No: 3, and a nucleotide sequence of the downstream primer is shown in SEQ ID No: 4.

The present disclosure provides an application of the heat-shock related gene ZmHsf11 in reducing or increasing the heat resistance of plants.

The regulation of heat resistance of the plant is to increase the heat resistance of the plant. Deletion or inhibition of the ZmHsf11 gene expression in the plant may increase the heat resistance of the plant.

The present disclosure provides a method for breeding a plant species resistant to high temperatures. The method includes knocking out or suppressing the expression of the heat-stress related gene ZmHsf11 to improve a survival rate of the plant after a heat treatment, such that the heat resistance of the plant may be improved.

The plant is rice or maize.

The present disclosure provides a heat-resistance negative-regulation related gene ZmHsf11 and a protein of the gene ZmHsf11. Specifically, in the present disclosure, the ZmHsf11 gene is cloned from the maize. An expression level of the ZmHsf11 gene can be induced by a high temperature and other adverse stresses. The ZmHsf11 gene may be overexpressed in rice. Functional detection is performed on the ZmHsf11 gene. It is found that, after overexpression, rice plants which has the overexpressed ZmHsf11 gene and receives a heat treatment has a significantly reduced survival rate. Functional detection is performed on a ZmHsf11 mutant gene provided by the present disclosure. It is found that, rice plants which has the ZmHsf11 mutant gene has significantly increased heat resistance. The above results indicate that the ZmHsf11 gene has negative regulation on the heat resistance of the plant. The present disclosure provides a new technical basis for breeding maizes for heat resistance.

### BRIEF DESCRIPTION OF THE DRAWINGS

FIG. 1A shows a chart of tissue-specific expression and an induced-expression pattern of the ZmHsf11 gene according to an Example 1 of the present disclosure.

FIG. 1B shows the induced-expression pattern of the ZmHsf11 gene in response to 42° C. according to the Example 1 of the present disclosure.

FIG. 1C shows the induced-expression pattern of the ZmHsf11 gene in response to 20% PEG according to the Example 1 of the present disclosure.

FIG. 1D shows the induced-expression pattern of the ZmHsf11 gene in response to 200 mM NaCl according to the Example 1 of the present disclosure.

FIG. 1E shows the induced-expression pattern of the ZmHsf11 gene in response to 100 μM ABA according to the Example 1 of the present disclosure.

FIG. 2 shows subcellular localization of the protein encoded by the ZmHsf11 gene according to an embodiment 2 of the present disclosure.

FIG. 3A shows GUS identification of rice positive strains having overexpression.

FIG. 3B shows semi-quantitative expression level of the rice positive strains according to an embodiment 3 of the present disclosure.

FIG. 4 shows phenotypes of the survival rate of the rice plants having the overexpression under the heat treatment according to an embodiment 4 of the present disclosure.

FIG. 5 shows a statistical chart of the survival rate of the rice plants having the overexpression under the heat treatment according to an embodiment 4 of the present disclosure.

FIG. 6 shows concentrations of the proline contained in the rice plants having the overexpression under the heat treatment according to an embodiment 4 of the present disclosure.

FIG. 7 shows DAB staining results of the rice plants having the overexpression under the heat treatment according to an embodiment 4 of the present disclosure.

FIG. 8 shows Trypan-blue staining results of the rice plants having the overexpression under the heat treatment according to an embodiment 4 of the present disclosure.

FIG. 9A shows the ZmHsf11 gene structure.

FIG. 9B shows PCR identification of a maize mutant (mu) variant according to an embodiment 5 of the present disclosure.

FIG. 9C shows RT-qPCR expression levels of the maize mutant (mu) variant according to an embodiment 5 of the present disclosure.

FIG. 10 shows phenotypes of the maize mutant (mu) variant under the heat treatment according to an embodiment 6 of the present disclosure.

FIG. 11 shows concentrations of the proline contained in the maize mutant (mu) variant under the heat treatment according to the embodiment 6 of the present disclosure.

FIG. 12 shows DAB staining results of the maize mutant (mu) variant under the heat treatment according to the embodiment 6 of the present disclosure.

### DETAILED DESCRIPTION

Technical solutions of the present disclosure are illustrated in details by referring to the embodiments. However, these embodiments are used for illustrative purposes only and do not limit the scope of the present disclosure.

It should be understood that the experimental methods used in the following embodiments are conventional methods if not specifically described.

It should also be understood that the experimental materials, reagents, and so on, used in the following embodiments are commercially available, if not otherwise specified.

A full length of a CDS sequence (SEQ ID No: 1) of the maize ZmHsf11 gene and an amino acid sequence (SEQ ID No: 2) of a protein encoded by the maize ZmHsf11 gene are obtained from the Plant Genome Database web site (https://phytozome.jgi.doe.gov/pz/portal.html).

A heat-shock related gene ZmHsf11 is provided by the present disclosure, and a nucleotide sequence of the heat-shock related gene ZmHsf11 is shown as SEQ ID NO: 1.

The present disclosure provides a protein encoded by the heat-shock related gene ZmHsf11, and the protein is described in (1) or (2) as follows.

(1) A protein consists of an amino acid sequence as shown in SEQ ID No: 2 in the sequence listing.

(2) A protein is derived from the protein (1) by substituting and/or adding one to ten amino acid residues to the amino acid sequence of SEQ ID No: 2 in the sequence listing, and has the function of the heat-shock related gene ZmHsf11.

The present disclosure provides a plant expression vector for the heat-shock related gene ZmHsf11. A nucleic acid molecule of the heat-shock related gene ZmHsf11 is inserted into an expression vector p1301a, and a vector for overexpressing the ZmHsf11, p1301a-ZmHsf11, is obtained.

The present disclosure provides a host bacterium of the plant expression vector.

The present disclosure provides a plant cell including the heat-shock related gene ZmHsf11, a plant including the plant cell, and a seed of the plant, and the plant is preferably rice or maize.

The present disclosure provides a primer pair for cloning the heat-shock related gene ZmHsf11. The primer pair includes an upstream primer and a downstream primer. A nucleotide sequence of the upstream primer is shown in SEQ ID No: 3, and a nucleotide sequence of the downstream primer is shown in SEQ ID No: 4.

The present disclosure provides an application of the heat-shock related gene ZmHsf11 in reducing or increasing the heat resistance of plants.

The regulation of heat resistance of the plant is to increase the heat resistance of the plant. Deletion or inhibition of the ZmHsf11 gene expression in the plant may increase the heat resistance of the plant.

The present disclosure provides a method for breeding a plant species resistant to high temperatures. The method includes knocking out or suppressing the expression of the heat-stress related gene ZmHsf11 to improve a survival rate of the plant after a heat treatment, such that the heat resistance of the plant may be improved.

The plant is rice or maize.

### Embodiment 1

### Analysis of a Tissue Expression Pattern and an Induced Expression Pattern of the ZmHsf11 Gene

### 1. Treatment on Maize Leaf

Maize B73 is planted in a pot having a diameter of 40 cm and a height of 50 cm. 3 maize seeds are planted in one pot, and the seeds are planted in 6 pots in total. The 6 pots are placed in a greenhouse (28° C. approximately) and grown for 14 days. Roots, stems and leaves of the maizes are sampled respectively from the 6 pots, snap frozen in liquid nitrogen, and stored at −80° C. When the maize is grown to reach a flowering stage, filaments, bracts and stamens of the maizes are sampled respectively, snap frozen in liquid nitrogen, and stored at −80° C. RNAs of all samples are extracted, reverse transcribed into cDNA samples, and stored in a refrigerator at −20° C.

In addition, the maize B73 is planted in a pot having a diameter of 15 cm and a height of 10 cm. 4 maize seeds are planted in one pot, and the seeds are planted in 24 pots in total. The 24 pots are placed in a greenhouse (28° C. approximately) and grown for 14 days. A treatment of 42° C., 200 Mm NaCl, 20% PEG6000 and 100 μM ABA is performed on each of the 24 pots. Samples are taken at 0 h, 1 h, 3 h, 6 h, 12 h and 24 h after the treatment. Each sample is mixed from three plants. All samples are snap frozen in liquid nitrogen and stored at −80° C. RNAs of all samples are extracted, reverse transcribed into cDNA samples, and stored at −20° C. in a refrigerator.

### 2. Primer Design for the Gene ZmHsf11

Based on a full length of a cDNA sequence of the obtained ZmHsf11 gene, RT-qPCR quantification primers are designed by using the Primer5 software.

A primer qPCR-ZmHsf11-F: 5′-CTGGGAGCGACCACGACG-3′.

A primer qPCR-ZmHsf11-R: 5′-AAACACTGGAGATTTTTACATAGG-3′.

A gene GAPDH of the maize is taken as an internal reference gene, serving as a control, and primers are designed.

A primer of Zm-GAPDH-F: 5′-CCTCTGGAAAA TTGTGGCGTG-3′.

A primer of Zm-GAPDH-R: 5′-GCCCAAACGAACAGTCAAGTC-3′.

[symbol]

### 3. RT-qPCR Reaction System and Procedures

### Reaction System

The qPCR is performed by diluting a concentration of the cDNA templates for 10 times

$$\begin{matrix}cDNA & 2 µL \\ forward primer & 1 µL \\ reverse primer & 1 µL \\ SYBR enzyme & 10 µL \\ RNA free Water & \underline{6 µL} \\ & 20 µL\end{matrix}$$

### Reaction Procedures

$$\begin{matrix}95 ° C . & 5 min \\ \begin{matrix}\begin{matrix}95 ° C . \\ 60 ° C .\end{matrix} \\ 60 ° C .\end{matrix} & \begin{matrix}10 s \\ 30 s \\ 1 min\end{matrix} \} 40 cycles\end{matrix}$$

Amplification signals and data are processed by applying a comparative Ct method (ΔΔCt method). A relative expression value Cq=2(−ΔΔCt). For each fluorescence quantification, three biological replicates and three technical replicates are performed.

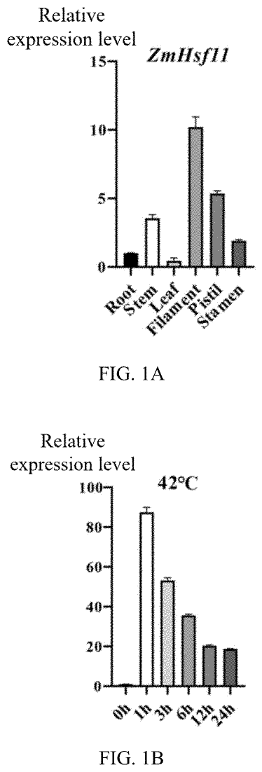

As shown in FIGS. 1A-1E, the ZmHsf11 gene is expressed in all tissues of the maize B73. However, expression levels in the stems, pistils and stamens of the maize are significantly higher than the expression levels in other tissues of the maize. When the heat treatment is performed, the expression level is increased significantly at 1 h after the heat treatment, and then decreased gradually as a heat-stress time length increases. When the high salt treatment, the osmotic treatment, and the ABA treatment are performed, the expression level is lower than that of the control sample at 0 h of the treatments. The above results suggest that the ZmHsf11 may be involved in abiotic stress pathways, such as the heat stress, the salt, the osmotic stress and the ABA.

### Embodiment 2

### Cloning and Subcellular Localization of the ZmHsf11 Gene

### 1. Primer Design:

By analyzing the CDS sequence of ZmHsf11 and a sequence of a subcellular localization vector p1305-GFP, following primers are designed.

A primer ZmHsf11-F (XbaI): 5′-gctctagaATGGCCGCCGAGCATGCCA-3′.

A primer ZmHsf11-R (SmaI): 5′-cccccgggCCTCGAGTCGTTGGACCC-3′.

### 2. Vector Construction

(1) The cDNA obtained in the Example 1 is taken as a template for PCR cloning and a reaction system is as follows.

$$\begin{matrix}template & 2 µL \\ F & 1 µL \\ R & 1 µL \\ 2 \times KOD mix & 12.5 µL \\ dd {H}_{2} O & \underline{8.5 µL} \\ & 25 µL\end{matrix}$$

### PCR Reaction Procedures:

$$\begin{matrix}98 ° C . & 3 min \\ \begin{matrix}\begin{matrix}98 ° C . \\ 60 ° C .\end{matrix} \\ 68 ° C .\end{matrix} & \begin{matrix}10 s \\ 5 s \\ 5 s\end{matrix} \} 35 cycles \\ 68 ° C . & 5 min\end{matrix}$$

At the end of the PCR reaction, 3 μL of 10× loading buffer is added. Later, agarose gel electrophoresis is performed. A gel extraction kit is applied to extract a target fragment.

(2) The PCR product extracted from (1) and an empty p1305-GFP vector are digested. A digestion system is as follows.

$$\begin{matrix}dd {H}_{2} O & 7 µL \\ PCR product / plasmid & 9 µL \\ Cut Smart Buffer & 2 µL \\ XbaI & 1 µL \\ SmaI & {1 µL}_{\_} \\ & 20 µL\end{matrix}$$

The reaction is carried out in a metal bath at 37° C. for 3 h. After the digestion is completed, 3 μL of loading buffer is added to terminate the reaction. Further, agarose gel electrophoresis is performed, and the digested product is extracted.

(3) Linkage reaction is performed, and a linkage reaction system is as follows:

$$\begin{matrix}Digestion products of PCR purified fragment & 6 µL \\ p 1305 - GFP digestion product & 2 µL \\ 10 \times T 4 buffer & 1 µL \\ T 4 DNA Ligase & \underline{1 µL} \\ & 10 µL\end{matrix}$$

The above linkage reaction system is mixed evenly, and placed into a PCR instrument. The reaction is performed at 16° C. for 1 h.

(4) A ligated product is transfected into E. coli and amplified to obtain a recombinant plasmid.

### 3. Protoplasmic Transformation.

Protoplasts of the maize B73 are obtained. The p1305-GFP-ZmHsf11 recombinant plasmids and a nuclear localization signal plasmid NLS-RFP are co-transfected into the protoplasts. The transfected protoplasts are incubated at dark for 18 h and then observed by a laser confocal microscopy.

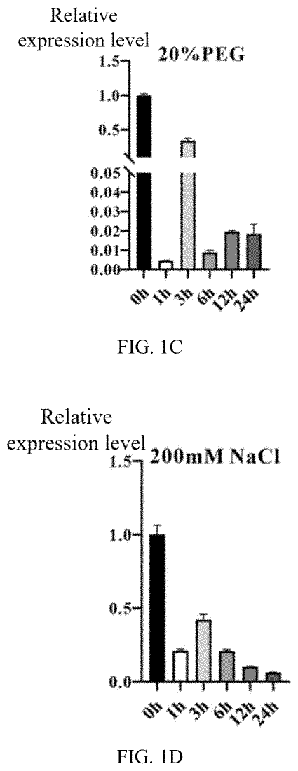

The results are shown in FIG. 2. The empty p1305-GFP vector is expressed on both the nucleus and the cell membrane, while the p1305-ZmHsf11:GFP shows green fluorescence only on the nucleus. The nuclear localization signal shows red fluorescence. Overlapping of the green fluorescence and the red fluorescence shows yellow fluorescence. The results suggest that the ZmHsf11 protein is a protein localized on the nucleus.

### Embodiment 3

### Obtaining Rice Plants Overexpressing the ZmHsf11 Gene

### 1. Similar to the Method in the Embodiment 2, an Overexpression Recombinant Plasmid is Obtained by Using the Following Primers, and the Recombinant Plasmid is Transfected Into Agrobacterium.

A primer ZmHsf11-F(KpnI): 5′-gggtaccATGGCCGCCGAGCATGCCA-3′.

A primer ZmHsf11-R(PstI): 5′-gctctagaTCACCTCGAGTCGTTGGACCC-3′.

### 2. Agrobacterium-Mediated Genetic Transformation Process of Rice Healing Tissues

Sterilized seeds are inoculated into an inoculation flask containing an induction medium. No more than 8 seeds are inoculated in one flask. The flask is incubated in a tissue culturing room for 14-20 days.

### (2) Infestation of the Healing Tissue

An Agrobacterium solution containing the target gene is scribed on a resistant YEP solid medium and incubated at 28° C. for 2 days. A single colony is picked and incubated in a 15 mL shaking tube containing 5 mL of media for 36 h. 3 mL of glucose, 800-900 μL of bacterial solution, and 150 μL of AS are added to 150 mL of suspension medium for expanding the culture.

The healing tissue is placed in a tissue culturing bottle, 50 mL of bacterial solution is added, and the bottle is shaken gently for 10 min. The operation is repeated once. The infested healing tissue is incubated on the solid YEP medium containing K+ and Rif, at 24° C., for 3 days. The amount of bacterial growth in the dish is observed. Sterilized ddH2O is used to wash the healing tissue for 5 min at a time, and the washing is repeated 5-7 times. Further, the washed healing tissue is transferred to a clean bottle and soaked in carboxybenzyl water for 30 min (performed separately twice). The healing tissue is dried on a filter paper, inoculate into a selection medium, placed in the tissue culturing room, and incubated for 2 weeks.

### (3) Differentiation of the Healing Tissue

A new healing tissue is selected from the selection medium, inoculated to the differentiation medium, placed in the tissue culturing room, and incubated to have a differentiated seedling.

### (4) Rooting Culture

When the seedling is grown in the differentiation medium, the seedling is transferred to a rooting medium in a tissue culturing flask, and cultured for 4 weeks.

### (5) Exercising the Seedling

Tap water is added to a rooting bottle, and the seedling is moved to the ground after 5 days in the rooting bottle.

### 3. Verifying Positive Rice Plants

### (1) GUS Staining

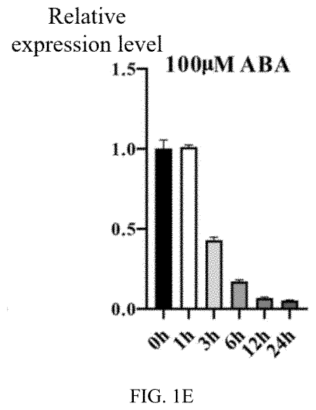

Rice leaves are sampled and placed in a PCR tube. 100 μL of GUS staining solution is added to the tube, and the tube is placed in a 37° C. incubator. The tube is incubated overnight and protected from light. Leaf infestation is observed (FIG. 3).

### (2) RT-PCR Method

RNAs are extracted from the transgenic rice and reverse transcribed into cDNAs as templates. A semi-quantitative assay is performed to observe whether the transgenic rice has a band, and brightness of the band.

The results are shown in FIG. 3A. The leaves, stem cuttings, and roots of the transgenic rice are stained blue, while a wild type rice is not stained. In addition, the expression of the ZmHsf11 gene in the overexpression plants is determined by RT-PCR, and the results are shown in FIG. 3B. No band is shown in the wild type rice, while bands are shown in all three strains of the overexpression plants. Therefore, these three strains are selected for further studies.

### Embodiment 4

### Verification of Heat Resistance in the ZmHsf11 Overexpression Rice Plants

### 1. Transgenic Rice Phenotyping Experiments

A T02 seed is harvested and placed in a pipette box. Appropriate amount of water is added to the pipette box. The pipette box is placed in a greenhouse at 28° C. for about 14 days. The plant is transplanted into a small round pot (D=14.8 cm, H=12 cm) and placed in the greenhouse at 28° C. for 15 days. The plant is then placed in the greenhouse at 45° C. for 22 h. Subsequently, the plant is restored to the room temperature and cultured in the greenhouse at 28° C. for 20 days. Phenotypes of the rice before and after the heat treatment are observed. Survival rates of wild-type rice and overexpression rice are counted respectively.

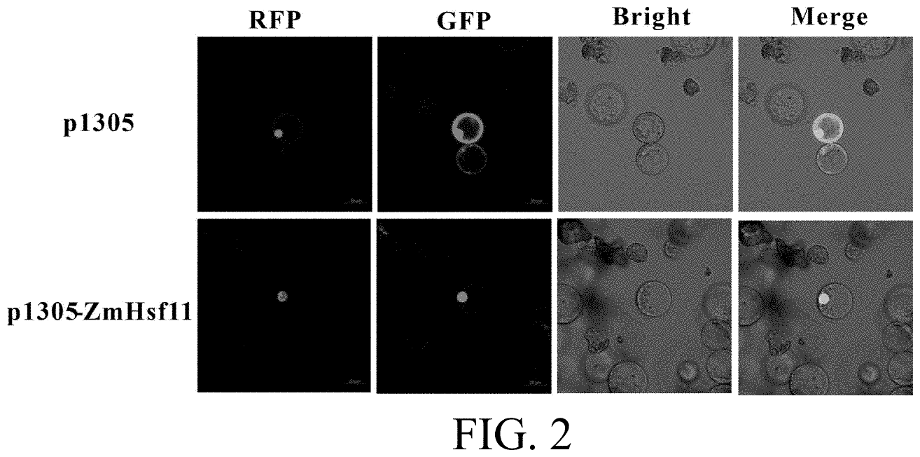

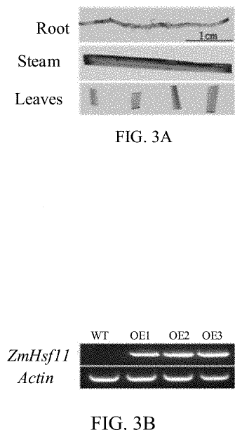

The results are shown in FIGS. 4 and 5. After the high temperature treatment and resumption of growth are performed, the survival rate of the overexpression plant is significantly lower than that of the wild type plant. The results indicate that overexpression of the ZmHsf11 gene in rice reduces the heat resistance of the plant.

### 2. Determination of Physiological and Biochemical Indicators

### (1) Proline Content Determination: Proline Contents in Rice Treated at 45° C. for 0 h, 45° C. for 1 h, and 45° C. for 6 h are Respectively Determined by a Kit.

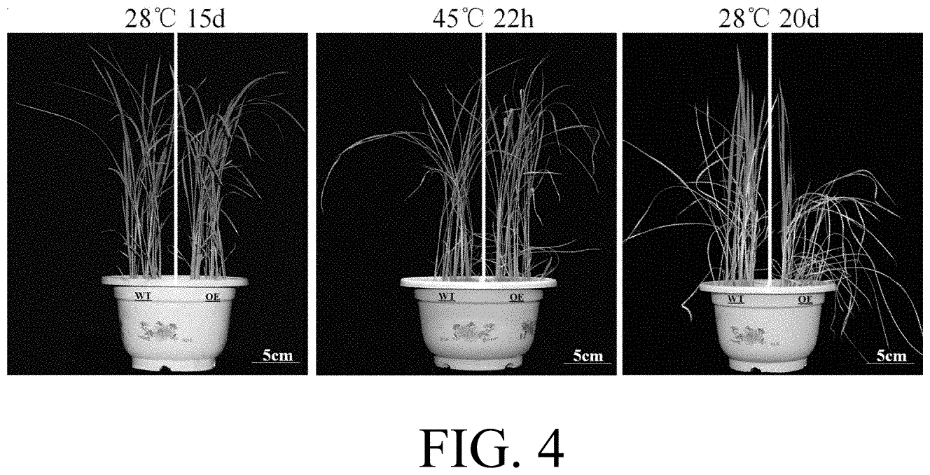

The results are shown in FIG. 6. At 0 h, the proline content of the overexpression plant is lower than that of the wild type plant, while at 1 hour after the heat treatment, the proline content of the overexpression plant is higher than that of the wild type plant. At 6 hours after the heat treatment the proline contents of the overexpression strains OE1 and OE2 are again lower than that of the wild type WT plant. The results suggest that the ZmHsf11 gene in rice may start functioning at 1 h after the heat treatment.

(2) DAB Staining and Trypan Blue Staining: The Rice Plant is Treated at 50° C. for 2.5 h. Subsequently, DAB Staining and Trypan Blue Staining are Performed on Plants Before and After the Treatment, and Staining Results are Observed.

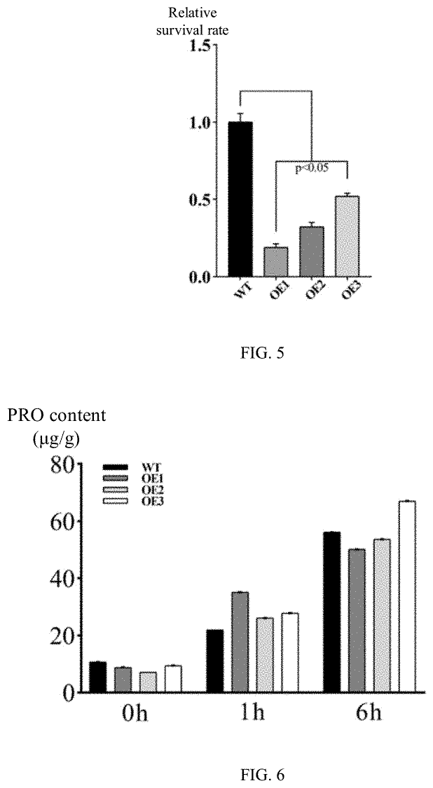

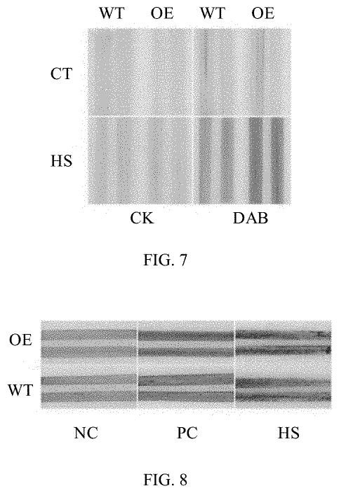

The results are shown in FIGS. 7 and 8. After the DAB staining, colour gradation and an area of the colour of the leaves of the overexpression plant are significantly higher than those of the wild type plant. After the Trypan Blue staining, an area of dead cells in the leaves of the overexpression plant is also significantly higher than that of the wild type plant. All these results indicate that overexpression of ZmHsf11 gene in the rice plant reduces the ability of overexpression plant responding to the high temperature stresses, suggesting that the ZmHsf11 gene may play a negative role in regulating the heat resistance of the plant.

### Embodiment 5

### Identification of ZmHsf11 Maize Mu Mutant Plant

The purchased maize species W22 is taken as a background for making a Mu mutant variant (from the Maize Genetics Cooperation Stock Center). The purchased maize species W22 is taken as a T0 generation. The T0 generation plant is verified by performing PCR, and a maize containing Mu transposons is screened. The screened maize containing the Mu transposons are self-interbred to obtain a T01 generation. A T02 generation is obtained by continuing performing the method described above. In this way, a homozygous seed is identified. The RT-qPCR is performed to determine the expression level of the ZmHsf11 gene in the mutant variant.

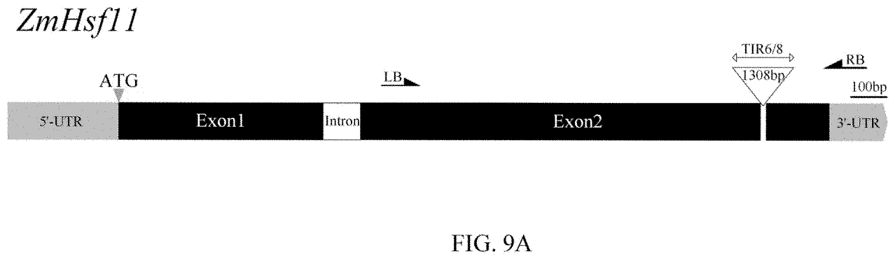

The results are shown in FIG. 9. The expression of the ZmHsf11 gene in the Mu mutant (numbered as Mu11 in the drawings) is lower than that in the wild type plant. The results suggest that the expression of the ZmHsf11 gene is suppressed or reduced in the Mu mutant, and the Mu mutant is a mutant losing the function of the ZmHsf11 gene.

### Embodiment 6

### Identification of Heat Resistance in the ZmHsf11 Mutant Maize Plant

### 1. Phenotypes of the Mutant

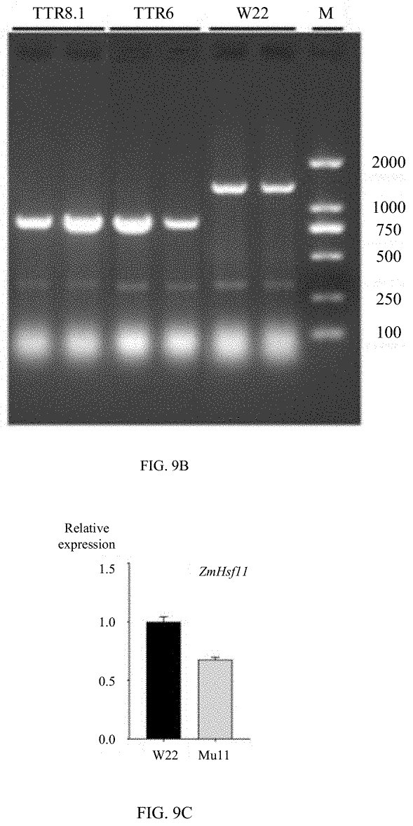

The seed of the wild-type W22 species and the Mu mutant are respectively seeded in a small round pot and grown in the greenhouse till the plants have three unfurled leaves and one new unfurled leaf. A moisture content of the pot of the wild-type W22 and a moisture content of the pot of the Mu mutant are controlled to be consistent with each other. The plants are placed in a light incubator and incubated overnight. Subsequently, the plants are placed in the greenhouse at 45° C. for 12 h. Later, the plants are placed at the room temperature and grown in the greenhouse for 2 days. Wilting of leaves of the wild-type plant and the Mu mutant are observed (as shown in FIG. 10).

The results are shown in FIG. 10. After the heat treatment, leaves of the wild-type plant are significant wilting, while leaves of the Mu mutant plant are not wilting. The results suggest that the Mu mutant plant is more resistant to heat than the wild-type plant is.

### 2. Determination of Physiological and Biochemical Indicators

### (1) Proline Content Determination: The Proline Contents in the Wild-Type Plant and in the Mu Mutant Plant at 0 h, 1 h and 6 h After a 45° C. Treatment are Determined by Using a Kit.

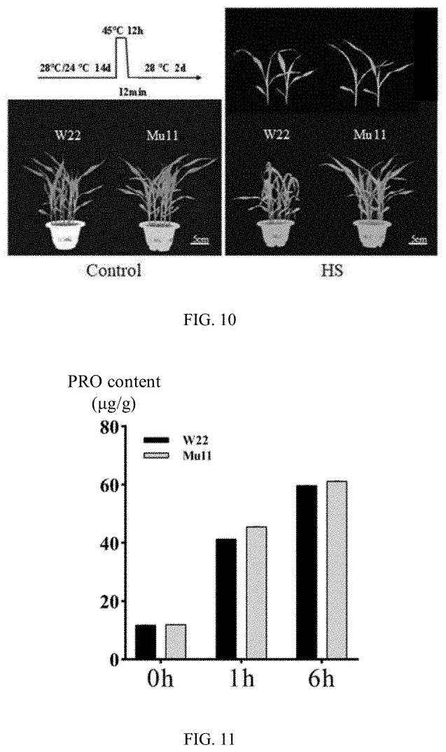

The results are shown in FIG. 11. The proline content increases sharply as a heat stress time length increases. In addition, for different treatment time lengths, the proline content of each Mu mutant is slightly higher or higher than that of wild-type plant.

### (2) DAB Staining

The wild-type plant and the Mu mutant plant are treated at 50° C. for 4 h. A 5 cm-tip of a third leaf of the wild-type plant before the 50° C. treatment, a 5 cm-tip of a third leaf of the wild-type plant after the 50° C. treatment, a 5 cm-tip of a third leaf of the Mu mutant plant before the 50° C. treatment, and a 5 cm-tip of a third leaf of the Mu mutant plant after the 50° C. treatment are obtained and stained by DAB, and staining results thereof are observed.

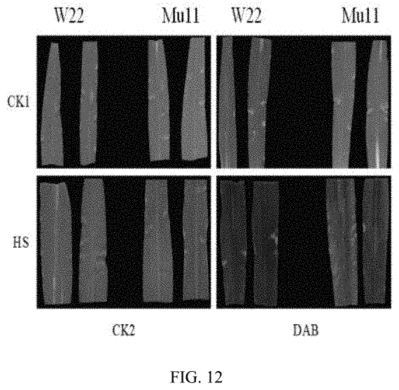

The results are shown in FIG. 12. After the DAB staining, colour gradation of the leaf of the Mu mutant plant is significantly lower than that of the wild-type plant. These results indicate that the loss of function of the ZmHsf11 gene enhances the heat resistance of the plant, further indicating that the ZmHsf11 gene plays a negative regulatory role in the heat resistance of the plant.

It shall be understood that the above description only shows preferred embodiments of the present disclosure and is not intended to limit the present disclosure. It shall be understood by any ordinary skilled person in the art that the present disclosure is not limited by the above embodiments. The above embodiments and the description describe only illustrative of the principles and methods of the present disclosure. Various variations and improvements may be made to the present disclosure without departing from the spirit and scope of the present disclosure. The scope of present disclosure is defined by the appended claims, the specification and their equivalents.

What is claimed is:

1. An application of a heat-stress related gene ZmHsf11 in reducing heat resistance of a plant, wherein the heat-stress related gene ZmHsf11 is overexpressed to reduce the heat resistance of the plant, and the heat-stress related gene ZmHsf11 has a nucleotide sequence of SEQ ID No: 1, and the plant is rice or maize.

2. An application of a heat-stress related gene ZmHsf11 in increasing heat resistance of a plant, wherein the heat-stress related gene ZmHsf11 is deleted or inhibited in the plant to increase the heat resistance of the plant, the heat-stress related gene ZmHsf11 has a nucleotide sequence of SEQ ID No: 1, and the plant is rice or maize.

3. A method of breeding a heat-resistant plant, comprising:
knocking out or suppressing expression of a heat-stress related gene ZmHsf11 to improve a survival rate of the plant after a heat treatment and to improve heat resistance of the plant;
wherein a nucleotide sequence of the heat-stress related gene ZmHsf11 is shown as SEQ ID No: 1, and the plant is rice or maize.
